(19) United States
(12) Patent Application Publication
(10) Pub. No.: US 2025/0205144 A1
(43) Pub. Date: Jun. 26, 2025

(54) SKIN TIGHTENING AND SMOOTHING PERSONAL CARE PRODUCTS

(71) Applicant: CARGILL, INCORPORATED, Wayzata, MN (US)

(72) Inventors: Dominique Karine Clio, Oplinter (BE); Pauline Darcel, Ixelles (BE); Jean-Noel Ollagnier, Evere (BE)

(73) Assignee: CARGILL, INCORPORATED, Wayzata, MN (US)

(21) Appl. No.: 18/851,800

(22) PCT Filed: Mar. 31, 2023
(86) PCT No.: PCT/US2023/017076

§ 371 Date: Sep. 27, 2024

Related U.S. Application Data
(60) Provisional application No. 63/325,776, filed on Mar. 31, 2022.
(60) Provisional application No. 63/374,729, filed on Sep. 6, 2022.
(60) Provisional application No. 63/378,887, filed on Oct. 10, 2022.

(51) Int. Cl. A61K 8/9717; A61Q 19/00

(52) U.S. Cl. CPC A61K 8/9717; A61Q 19/00

(57) ABSTRACT

The present invention relates to the production of personal care products that have desirable texture and viscosity as well demonstrating skin tightening, smoothing, or cooling properties. These personal care products contain a specific mixture of carrageenans to yield these desired properties. More specifically, a topical formulation comprising a carrageenan component wherein the carrageenan component comprises between 7 to 12 wt % kappa type carrageenan; between 15 to 25 wt % iota type carrageenan; between 60 to 80 wt % lambda type carrageenan; and wherein the carrageenan component comprises between 0.2 and 3 wt % of the topical formulation is described.

### CROSS-REFERENCE TO RELATED APPLICATIONS

This application claims the benefit of U.S. Provisional Application No. 63/325,776, filed Mar. 31, 2022, U.S. Provisional Application No. 63/374,729, filed Sep. 6, 2022, and U.S. Provisional Application No. 63/378,887, filed Oct. 10, 2022, each of which is incorporated by reference herein in its entirety.

### FIELD OF INVENTION

The present invention relates to the production of personal care products that have desirable texture and viscosity as well demonstrating skin tightening, smoothing, or cooling properties. These personal care products contain a specific mixture of carrageenans to yield these desired properties.

### BACKGROUND

Carrageenans are a family of natural linear sulphated polysaccharides that are typically extracted from red edible seaweed. The most well-known and still most important red seaweed used for manufacturing of carrageenan is Chondrus crispus (Irish moss) which is a dark red parsley-like plant that grows attached to the rocks. Carrageenans are large, highly flexible molecules that form curling helical structures in solutions or mixtures. This gives them the ability to form a variety of different gels at room temperature and they are are widely used in the food industry, for their gelling, thickening, and stabilizing properties, however, they have also gained interest in many other applications because of these properties. They are known to be effective ingredients in many personal care applications.

While other forms exist such as mu and nu, there are three main commercial classes of carrageenan (kappa, iota, and lambda). Kappa forms strong, rigid gels in the presence of cations such as sodium, potassium, and calcium ions, and reacts with dairy proteins. It is sourced mainly from Kappaphycus alvarezii. Iota forms soft gels in the presence of calcium ions. It is produced mainly from Eucheuma denticulatum. Lambda forms do not gel and are often used to thicken dairy products.

The primary differences influencing the properties of the carrageenans, including kappa, iota, and lambda carrageenan, are the number and position of the ester sulfate groups on the repeating galactose units. Higher levels of ester sulfate lower the solubility temperature of the carrageenan and produce lower strength gels, or contribute to gel inhibition (eg. lambda carrageenan).

All are soluble in hot water, but in cold water, only the lambda form (and the sodium salts of the other two) are soluble.

### SUMMARY OF INVENTION

While the uses of the various forms of carrageenan are known in a broad variety of commercial settings, each of the forms has positive and negative functional attributes. It would be desirable to have formulations that could take advantage of the collective positive attributes of the various forms without suffering the negatives. However, because of the varying properties of the different forms of carrageenans, predicting the properties of any mixture of the forms is very difficult.

The present disclosure relates to surprising discovery that specific blends of kappa, lambda, and iota type carrageenan have a highly desirable ability to tighten and smooth the skin.

In one aspect the present invention discloses a topical formulation comprising a carrageenan component wherein the carrageenan component comprises:
a) between 7 to 12 wt % kappa type carrageenan:
b) between 15 to 25 wt % iota type carrageenan:
c) between 60 to 80 wt % lambda type carrageenan; and
wherein the carrageenan component comprises between 0.2 and 3 wt % of the topical formulation.

Another aspect discloses a method of tightening or smoothing the skin of a human comprising applying to the skin a topical formulation comprising a carrageenan component wherein the carrageenan component comprises:
a) between 7 to 12 wt % kappa type carrageenan:
b) between 15 to 25 wt % iota type carrageenan:
c) between 60 to 80 wt % lambda type carrageenan; and
wherein the carrageenan component comprises between 0.2 and 3 wt % of the topical formulation.

Another aspect discloses a method of tightening the skin of a human comprising applying to the skin a topical formulation comprising a carrageenan component wherein the carrageenan component comprises:
a) between 7 to 12 wt % kappa type carrageenan:
b) between 15 to 25 wt % iota type carrageenan:
c) between 60 to 80 wt % lambda type carrageenan; and
   wherein the carrageenan component comprises between 0.2 and 3 wt % of the topical formulation.

Another aspect discloses a method of smoothing the skin of a human comprising applying to the skin a topical formulation comprising a carrageenan component wherein the carrageenan component comprises:
a) between 7 to 12 wt % kappa type carrageenan:
b) between 15 to 25 wt % iota type carrageenan:
c) between 60 to 80 wt % lambda type carrageenan; and
   wherein the carrageenan component comprises between 0.2 and 3 wt % of the topical formulation.

Another aspect discloses a method of cooling the skin of a human comprising applying to the skin a topical formulation comprising a carrageenan component wherein the carrageenan component comprises:
a) between 7 to 12 wt % kappa type carrageenan:
b) between 15 to 25 wt % iota type carrageenan:
c) between 60 to 80 wt % lambda type carrageenan; and
   wherein the carrageenan component comprises between 0.2 and 3 wt % of the topical formulation.

In another aspect the present invention is directed to a blend of carrageenans comprising:
a) between 7 to 12 wt % kappa type carrageenan:
b) between 15 to 25 wt % iota type carrageenan; and
c) between 60 to 80 wt % lambda type carrageenan.

### DETAILED DESCRIPTION

Explanations of abbreviations and terms used in this disclosure are provided to assist in comprehending and practicing the invention.

All ratios of composition, blend, or formulation components referred to herein by “percentage” means percentage by weight (wt %), unless otherwise specifically specified.

All parameter ranges disclosed include the end-points and all values in between, unless otherwise specified.

Representative features are set out in the following description, which stand alone or may be combined, in any combination, with one or more features disclosed elsewhere in the description and/or drawings of the specification.

When used in this specification and claims, the terms “comprises” and “comprising” and variations thereof mean that the specified features, steps or integers are included. The terms are not to be interpreted to exclude the presence of other features, steps or components.

### Carrageenans

The basic structure of carrageenans is a linear polysaccharide made up of a repeating dissacharide sequence of β-D-galactopyranose linked through positions 1,3 (A residues) and α-D-galactopyranose residues linked through positions 1,4 (B residues). The regular backbone structure of the basic structure of carrageenans is disrupted by a more or less ordered distribution of sulphate groups. Some of the galactose units have attached sulfate groups, while others are unsulfated. The three main types (iota, kappa and lambda) of carrageenan molecules differ by (1) the types of linkages between the galactose units, and (2) the point of attachment and number of the sulfate groups on each of the galactose units. These apparently small differences in chemical constitution and structure make major differences in the properties of each type of molecule.

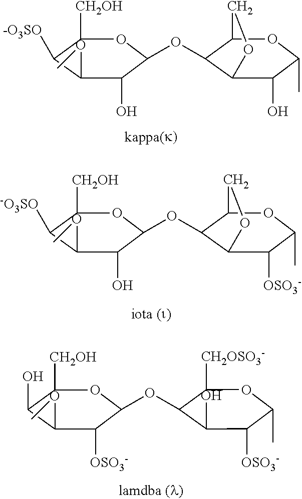

Gelling in carrageenan is caused by helix formation and this can only occur in repeat structures where the B residue is in a 1-C-4 conformation. Lambda carrageenan (theoretically having 3 sulphate groups per repeating unit) has both its sugar residues in a 4-C-1 conformation and therefore does not form gels. It is therefore known to the skilled person that lambda carrageenan should be avoided when gelling is a strong requirement of the end use of a formulation including carrageenans.

All the gelling types of carrageenan which include the kappa type (theoretically having 1 sulphate group per repeating unit: the most naturally abundant type of carrageenan molecule) and the iota type (theoretically having 2 sulphate groups per repeating unit: the least naturally abundant type of carrageenan molecule) all contain a 3,6-anhydro bridge on the B unit which forces the sugar to flip from a 4-C-1 conformation to a 1-C-4 conformation and can then form cross-link networks and gels.

Some types of seaweed species contains relatively pure carrageenan fractions. Kappaphycus Alvarezii contains largely kappa carrageenan and mu carrageenan which may be converted to kappa carrageenan by alkali treatment. Eucheuma Denticulatum contains a similarly high level of iota carrageenan with some nu carrageenan precursor. Other seaweeds are more mixed in their carrageenan content. Furcellaran contains a strong gelling type carrageenan which is a mix of kappa carrageenan and β carrageenan in a roughly 3:2 ratio. Other seaweed types, such as Chondrus crispus and Gigartina types contain not only a mix of kappa and lambda type carrageenans but also a type of carrageenan polymer that is essentially a block copolymer of different carrageenan types. This gives the carrageenan made from Gigartina or Chondrus weed species quite different properties from those made from the Eucheuma type species from Southeast Asia.

Many red algal species produce different types of carrageenans during various stages of their developmental history. For instance, the genus Gigartina produces mainly kappa carrageenans during its gametophytic stage, and lambda carrageenans during its sporophytic stage.

Carrageenan isolated from natural seaweed sources will contain long polymers substituted with a varying number of sulphate structures. Carrageenan will not be purely of one type of repeating units. For that reason, an isolated carrageenan can be more or less of one “type” based on the degree of sulphate substitution and differing linkages. A skilled artisan will appreciate the genus of seaweed, location of harvest, and stage of development will affect the type of carrageenan isolated. Further, a sample of carrageenan can be analysed by methods known in the art to determine what proportion of the polymers' monomeric residues are of what type. (see e.g. Aguibal et. al., Botanica Marina, Vol 46, (2003), pp 179-192; and Knutsen et. al., Carbohydrate Research, 331, (2001), 101-106.)

The term “Kappa Type” as used herein refers to a form of carrageenan that contains greater than 80% of its monomeric residues in the kappa form. In some aspects, kappa type will contain greater than 85% of its monomeric residues in the kappa form. In some aspects, kappa type will contain between 80% and 95% its monomeric residues in the kappa form.

The term “Iota Type” as used herein refers to a form of carrageenan that contains greater than 80% of its monomeric residues in the iota form. In some aspects, iota type will contain greater than 85% of its monomeric residues in the iota form. In some aspects, iota type will contain between 80% and 95% its monomeric residues in the iota form.

The term “Lambda Type” as used herein refers to a form of carrageenan that contains greater than 20% of its monomeric residues in the lambda form. In some aspects, lambda type will contain greater than 24% of its monomeric residues in the lambda form. In some aspects, lambda type will contain between 15% and 30% its monomeric residues in the lambda form.

Various forms of carrageenans are commercially available from a variety of commercial producers. For example, the carrageenans utilized in the present disclosure are commercially available from Cargill incorporated under the trade designations (VPC 614 kappa type, VPC 430 lambda type, and VPC 508 iota type). Similar to any natural product, carrageenan is a natural product isolated from cultivated or harvest seaweed, the exact content of any carrageenan extract will vary slightly depending on time of harvest or season.

TABLE 1
Compositions of commercially available
carrageenans from Cargill Incorporated

| | VPC 614 | VPC 430 | VPC 508 |
|---|---|---|---|
| | (kappa) | (lamda) | (iota) |
| Kappa* | 86.2 | 27.9 | 3 |
| Iota* | 9.8 | 21.3 | 86.2 |
| Lambda* | ND | 24 | ND |
| Mu* | 2.9 | 5.5 | 5.6 |
| Nu* | ND | 19.9 | ND |

*Values in the table represent an average content from 7 or more samples. ND means amounts of this form was not detected.

### Personal Care Composition

In one aspect, the present invention is the use of carrageenan mixtures disclosed herein in various personal care products. The personal care composition may be prepared by creating an aqueous phase of predominately water and adding the carrageenans with mixing. The mixing is often done at elevated temperature (approximately 75° C.) to facilitate complete incorporation. The type of mixing is not critical and typical mixing equipment used in the art may be employed with the present compositions. In order facilitate quicker and more efficient mixing, medium or high shear equipment is desirable.

The carrageenans may be added as a premix or individually. Additional ingredients may then be added to the formulation and pH adjusted as desired. The mixture is cooled with or without stirring to yield the final personal care composition. The personal care composition my contain other active or inactive ingredients, humectants, or emollients. Active ingredients are those having a physiological effect on the skin or hair. The composition may be an emulsion containing emollients, colorants, active ingredients, and/or other texturizers.

With the temperature still above the gelling point, pH may be adjusted to approximately 5.5 to 6.0 by addition of an acid such as citric acid. Additional salts or electrolytes are added to the mixture as preservatives and/or to manage the gelling properties of the carrageenans. The amount of salts or electrolytes may be varied according the texture sought to be achieved. Typically, the amount of salts or electrolytes is approximately 0.25% to 1% of the composition. In some aspects it is approximately 0.5%. The mixture may then be cooled. Typically, high shear mixing is avoided after the addition salts or electrolytes and upon cooling to preserve the final product texture.

The amount of carrageenans utilized the personal care formulation can be varied by the skilled artisan to achieve the desired texture. In some aspects the amount of carrageenans is between 0.2% and 3%. In other aspects the amount is between 0.5% and 2%. In another aspect the percentage of carrageenans in the personal care formula is between 0.5% and 1.5%.

### 1. Aqueous Phase

The topical formulation may contain an aqueous phase. The aqueous phase may comprise or consist of water, in particular a demineralized water: a floral water such as cornflower water: a mineral water such as Vittel water, Lucas water or La Roche Posay water; and/or a spring water. In some aspects, demineralized water, floral water, or a combination of both is used as the aqueous phase utilized by the present invention.

In some aspects, the amount of the aqueous phase in the topical formulation may be between 50 wt % to 99.5 wt %. In some aspects the amount of the aqueous phase in the topical formulation may be between 90 wt % to 98 wt %. The aqueous phase may optionally contain other soluble components such as an alcohol or polyol. In some aspects the aqueous phase may contain glycerol. In some aspects the topical formulation contains 1 to 4 wt % glycerol.

### 2. Emulsions

In some aspects, the personal care formulation may be an emulsion. An emulsion may be defined as a mixture containing two immiscible liquids, in which one liquid is dispersed as droplets or globules throughout the other. The dispersed liquid is called the dispersed phase, while the other liquid is called the continuous phase. In an oil-in-water emulsion, as in the present invention, the oil is the dispersed phase or oil phase, and water is the continuous phase or aqueous phase.

The topical formulation may also contains an oil phase dispersed in the aqueous phase. As used herein, the term “dispersion” refers to an oil phase forming droplets inside the aqueous phase. The droplets may have any sizes and shapes. Preferably, the droplets are homogeneously distributed throughout the aqueous phase. The nature of the oil phase of the emulsion is not critical. The oil phase may thus consist of any fatty substance conventionally used in the cosmetic or dermatological fields: in particular the oil phase may comprise at least one oil, i.e., any fatty substance that is in substantially or completely liquid form at room temperature (20-25° C.) or elevated temperate of (40-70° C.) and at atmospheric pressure (760 mmHg).

The preferred oil phase(s) comprises at least one oil which can be a hydrocarbon-based oil, i.e. an oil mainly containing hydrogen and carbon atoms and optionally oxygen, nitrogen, sulfur and/or phosphorus atoms, for example in the form of hydroxyl or acid radicals: a silicone oil, i.e. an oil comprising at least one silicon atom and preferably at least one Si—O group: a fluoro oil, i.e. an oil comprising at least one fluorine atom: a non-fluoro oil, or a mixture thereof.

The hydrocarbon-based oils may be of animal origin or of vegetable origin, such as liquid triglycerides of fatty acids comprising from 4 to 20 carbon atoms, examples include, coconut oil, canola oil, rapeseed oil, sunflower oil: maize oil: soybean oil: cucumber oil: grape seed oil: sesame seed oil: hazelnut oil: apricot oil: macadamia oil; arara oil: castor oil: cocoa butter: almond oil: avocado oil: babassu oil: caprylic/capric acid triglycerides, such as those sold by Stearineries Dubois or those sold under the names Miglyol 810, 812 and 818 by Dynamit Nobel: Simmondsia Chinensis (Jojoba) Seed oil sold under the tradename Jojoba Oil Golden by Desert Whale; Beta-carotene sold under the tradename Betatene 30% OLV by Cognis (BASF); Rosa Canina Fruit Oil sold under the tradename Rosehip Seed Oil by Nestle World Trade Co.; shea butter oil; and mixtures thereof.

The hydrocarbon-based oils may be linear or branched hydrocarbons of mineral or synthetic origin. Alternatively, the hydrocarbon-based oils may be synthetic ethers; synthetic esters; fatty alcohols that are liquid at room temperature, with a branched and/or unsaturated carbon-based chain containing from 12 to 26 carbon atoms; C12-C22 higher fatty acids; or mixtures thereof.

The personal care composition may further comprise at least one further ingredient. The further ingredient may include, without limitation, a preservative, salt, vitamin, emulsifier, texturizer, nutrient, micronutrient, sugar, protein, polysaccharide, polyol, glucose, sucrose, glycerol, sorbitol, pH adjusters, emollients, dyes, pigments, skin actives, waxes or silicones.

### Topical Formulation

The ingredients provided herein are useful in the manufacture of topical formulations such as personal care products or cosmetics. The inventors unexpectedly found that formulations comprising a combination of specific ratio of ratios of carrageenans have numerous desirable characteristics as explained further below.

In one aspect, the present invention is a topical formulation comprising an emulsion as described herein. As used herein, the term “topical formulation” refers to a formulation that may be applied directly to a part of the body. The term “formulation” is used herein to denote compositions of various ingredients in various weight ranges, in accordance with the present invention.

The formulations manufactured with the ingredients described herein are suitable for use on (and may be applied to) hair, scalp, nails and skin, for delivering cosmetic or actives to the skin or hair for providing cleansing, conditioning, moisturizing, minimizing or treating skin imperfections, reducing skin oiliness, providing fragrances to the hair or skin and the like.

“Personal care” means and comprises any cosmetic, hygienic, toiletry and topical care products including, without limitation, leave-on products (i.e., products that are left on keratinous substrates after application): rinse-off products (i.e., products that are washed or rinsed from keratinous substrates during or within a few minutes of application): shampoos: hair curling and hair straightening products: hair style maintaining and hair conditioning products: lotions and creams for nails, hands, feet, face, scalp and/or body: hair dye: face and body makeup: nail care products: astringents: deodorants: antiperspirants: anti-acne: antiaging: depilatories: colognes and perfumes: skin protective creams and lotions (such as sunscreens); skin and body cleansers: skin conditioners: skin toners: skin firming compositions: skin tanning and lightening compositions: liquid soaps: bar soaps: bath products: shaving products; and oral hygiene products (such as toothpastes, oral suspensions, and mouth care products).

The texture of such personal care formulations is not limited and may be, without limitation, a liquid, gel, spray, emulsion (such as lotions and creams), shampoo, pomade, foam, tablet, stick (such as lip care products), makeup, suppositories, among others, any of which can be applied to the skin or hair or hale and which typically are designed to remain in contact therewith until removed, such as by rinsing with water or washing with shampoo or soap. Other forms could be gels that can be soft, stiff, or squeezable. Sprays can be non-pressurized aerosols delivered from manually pumped finger-actuated sprayers or can be pressurized aerosols such as mousse, spray, or foam forming formulation, where a chemical or gaseous propellant is used.

The topical formulation comprising the emulsion disclosed herein may be a lotion, serum, or cream. Advantageously, the serum or cream may comprise specific ratio of ratios of lamda, kappa, and iota type carrageenans.

Formulations prepared using the ingredients disclosed herein have a clear colorless or light white color that is generally considered to be aesthetically appealing. In some cases, the formulations of the invention may be further processed to make a colored end product. In such cases, the lack of color is beneficial because it will show up the additional pigment without influencing the final color.

Furthermore, formulations prepared using the ingredients of the present invention have a good spreadability with pleasant residual feeling on the skin. The use of the ingredients of the present invention improve texture and body of personal care products. This texture feels pleasant to touch and to apply. Furthermore, the consistency is such that good product pick-up may be achieved. Good product pick-up means that sufficient product (i.e. not too much, and not too little) can be collected on the user's finger.

Personal care products used for tightening the skin can be applied by spreading the product onto the skin with the hand or by use of a brush, swab, cotton pads (rounds or squares), or a cloth. The frequency of use can be determined by the user and application could be daily or multiple times per day.

In one aspect, the ingredients of the present disclosure are particularly useful in sunscreen applications. Sunscreens contain ingredients intended to block UV radiation from reaching the skin. UV blockers can be physical such as or chemical salts like ZnO or TiO2 or chemical (max authorized level indicated) such as Butyl Methoxy dibenzoylmethane (5%); Octocrylene (10%); Titanium dioxide (25%); Ethylhexyl Salicylate (5%); Ethylhexyl Methoxy cinnamate (10%); Bis-ethylhexy loxyphenol Methoxyphenyl Triazine (10%); Emulsions of the present disclosure can be used with any type of UV blocker know in the art or mixtures of UV blockers.

### Blend

As used herein, the term “blend” refers to a physical mixture of two or more substances. In one aspect the present invention comprises a blend of carrageenans useful for preparations of the personal care composition described herein. In another aspect the present invention is directed to a blend of carrageenans comprising: between 7 to 12 wt % kappa type carrageenan; between 15 to 25 wt % iota type carrageenan; and between 60 to 80 wt % lambda type carrageenan. In another aspect, between 8 to 12 wt % kappa type carrageenan; between 17 to 23 wt % iota type carrageenan; and between 65 to 75 wt % lambda type carrageenan. In another aspect, these blends are used to make a personal care formulation or topical formulation and such formulations may be used to tighten or smooth the skin.

In addition, the personal care compositions and methods of the present invention may be manufactured using a blend carrageenans comprising or consisting of between 23 to 34 wt % kappa carrageenan; between 29 to 37 wt % iota carrageenan; between 12 to 20 wt % lambda carrageenan; between 2 to 8 wt % mu carrageenan; and between 12 to 19 wt % nu carrageenan.

The carrageenan blend can additionally contain other dry ingredients if desired in the final personal care composition such at texturizers, colorants, favors, fragrances, and the like. In one aspect the blend further comprises a texturizer. Texturizers including but not limited to cellulose and its derivatives, synthetic polymers like acrylates and carbomer, scleroglucan, and xanthan gum are useful in the present invention. Texturizers can be present in any useful or desired amount.

The blend can be prepared according to blending methods for dry ingredients that are known in the art. Use of a standard mixing equipment for a sufficient time will easily prepare the blends described herein.

In another aspect the present invention is directed to the use of individual types of carrageenans or any blend of the individual types of carrageenans in a personal care composition useful for the smoothing or tightening of the skin. Blends may include all types of carrageenans or just two or more of the types. In another aspect, these blends are used to make a personal care formulation or topical formulation and such formulations may be used to tighten or smooth the skin.

Application of carrageenans to the skin has surprisingly been shown to affect the overall topography of the skin surface. Despite a long history of use in personal care compositions to modify the texture and viscosity of products, this effect on the skin has never been reported. The skin topography is affected in two different ways. First, the product's application has been shown to reduce the height of small peaks and the depth of microfolds on the skin. Thereby demonstrating a smoothing effect where the skin's roughness is reduced. In addition, the product's application has been shown to narrow the width of microfolds in the skin thereby tightening the skin and eliminating or reducing the visibility of these microfolds. This effect could also be called the reduction of wrinkles or the reduction of fine lines or the reduction of crow's feet around the eyes. The term tightening would encompass any of these alternative descriptions or any description that embodies the concept of reducing the width of microfolds on the skin.

### Example 1

| | Amount | |
|---|---|---|
| Materials | Wt % | Source |
| Demineralized Water | Q.S 100 | |
| Rosa Damascena Flower Water | 60 | Aroma-Zone |
| VPC 430 (lamda carrageenan) | .70 | Cargill Incorporated |
| VPC 508 (iota carrageenan) | .20 | Cargill Incorporated |
| VPC 614 (kappa carrageenan) | .1 | Cargill Incorporated |
| Glycerol | 2.5 | Cargill Incorporated |
| Euxyl K712 | 1% | Schülke & Mayr GmbH |
| Citric Acid 50% aqueous | 0.05 (Q.S. | Merck |
| solution | pH 5.5) | |

Step 1: The aqueous phase of demineralized water and rose water was prepared and heated to approximately 70° C.
Step 2: Carrageenans were added slowly while mixing was continued and after addition the solution mixed for 5 minutes with a UltraTurrax set at 5000 rpms.
Step 3: The glycerol was added and the mixture was stirred for 3 minutes with a UltraTurrax set at 5000 rpms.
Step 4: The aqueous potassium sorbate and sodium benzoate was added the stirring continued until a homogenous mixture obtained. The pH was adjusted to between 5.0 and 5.5 by addition of 50% citric acid solution.

The resulting product was a slightly vicious gel that could still be easily pumped. It has a pleasant watery break upon application.

### Example 1A

Example 1A was produced in the same fashion as Example 1 except for the use of 2% Carrageenan blends instead of 1%.

### Example 2

| Materials | Amount Wt % |
|---|---|
| Demineralized Water | Q.S 100 |
| VPC 430 (lamda carrageenan) | .70 |
| VPC 508 (iota carrageenan) | .20 |
| VPC 614 (kappa carrageenan) | 1 |
| Aq. Potassium Sorbate/Sodium Benzoate | 0.15%/0.30% |
| Citric acid 50% aqueous solution | Q.S. pH 5.5 |

The process of Example 1 was followed (absent the addition of glycerol) to make the product of Example 2. This slightly viscous gel was evaluated for its ability to smooth and tighten human skin. Tightening and smoothing evaluation was performed with Polytec TMS-500 light interferometer using topography analysis. Polytec TMS 3.8 software was used to evaluate the surface microrelief of the skin explants before and 10 minutes after a single application (2 mg/cm²) of the composition of Example 2. Skin explants contain a measurable topography of high and low points as well as deeper microfolds. In evaluating smoothing the polytech instrument is used to measure the change in high and low points of the skin sample after application of the test compound. In evaluating tightening effect, the instrument is used to measure the width of microfolds in the skin sample before and after application of the test compound.

### Results

After application of the sample of Example 2, there was a surprising and immediate improvement in skin smoothness of 11%. In addition, the median width of microfolds was reduced from 147 μm to 129 μm (11%) demonstrating a tightening effect. In addition, the tightening affect was observed by 74% of individuals in a sensory panel. This formula can therefore be used as a visual eraser to visibly smooth the skin and reduce fine lines.

Upon application of Example 1A, tightening effects were evident after 3 hours (10% reduction in microfold depth and 12.8% improvement in skin smoothness) and after 6 hours (6.6% reduction in microfold depth and 8.8% improvement in skin smoothness).

Application of Example 1 and 1A were shown to improve skin firmness. The firmness of skin can be measured with a commercially available device from Dynaskin. The Dynaskin will blow air either perpendicular or at a 45 degree angle to the skin which creates a reproduceable and measurable deformation in the skin surface. Measurements before and after application of a test compound can evaluate any effect on the firmness of the skin.

Ten minutes after application of a sample of Example 1, firmness of the skin was improved such that the volume of deformation was reduced 5.6% and the depth of deformation was reduced 5.9%, 10 minutes after application of a sample of Example 1A, firmness of the skin was improved such that the volume of deformation was reduced 10.7% and the depth of deformation was reduced 6.6%. The study was repeated for Example 1, but measurements were done at 3 and 6 hours post application. At the 3 hour time point, the volume of deformation was reduced 10.7% and the depth of deformation was reduced 9.7%. At the 6 hour time point, the volume of deformation was reduced 7.8% and the depth of deformation was reduced 8.4%.

The study was repeated for Example 1A, measurements were done at 3 and 6 hours post application. At the 3 hour time point, the volume of deformation was reduced 11.9% and the depth of deformation was reduced 7.1%. At the 6 hour time point, the volume of deformation was reduced 10% and the depth of deformation was reduced 6.3%.

Importantly, these effects were observed with a serum formula with pleasant texture, stability, and spreadability. Use of kappa type carrageenan alone is a challenge. Typically, creating an acceptable formulation with kappa type carrageenan alone is difficult because at levels necessary to thicken the formula syneresis becomes a problem. Syneresis is the contraction of a gel with concomitant separation of liquid. In the case of kappa type carrageenan, at elevated concentrations the gel will experience syneresis over time. For this reason, it is unfavorable to use kappa type carrageenan alone.

In an additional aspect, the carrageenans disclosed herein have been shown to have surprisingly cooling effect when applied to the skin. A clinical evaluation of the cooling effect was done by application of test samples to the forearms of 20 Caucasian female volunteers between the ages of 22 and 77. Each individual had 4 testing areas identified, two on each arm. One area was not treated as thus maintained as the control. Three samples were applied (2 μl/cm²) to the other areas by finger until absorbed into the skin. Skin temperature was monitored by infrared camera for 15 min. The three samples included Example 1, Example 1A. and a commercial benchmark sold on the market for cooling. The benchmark sample had a maximum lowering of the skin temperature of 2.4° C. Example 1A also showed a maximum temperature lowering of 2.4° C. while Example 1 showed a maximum temperature lowering of 2.7° C. Surprisingly, both Examples 1 and 1A showed a long duration of cooling and greater overall cooling effect than the commercial benchmark.

### Stability

Stability is evaluated visually by observing the stability of the personal care formulation over time. The formulation fails if the phases separate or begin to separate. Major pooling of oil or water droplets on the surface or a significant change in the visual color or texture of the samples would also indicate failure of the formulation. In addition, viscosity could be rechecked and significant decrease (>20%) would indicate failure of the formulation.

The carrageenan containing formulations disclosed herein have good stability, with little or no breakdown or separation between phases over an extended period of time (e.g., 4, 8, or 12 weeks) when measured at room temperature and at 45° C. Therefore, it may be used to make products (e.g., topical formulations) requiring a long shelf life.

1. A topical formulation comprising a carrageenan component wherein the carrageenan component comprises:
a) between 7 to 12 wt % kappa type carrageenan;
b) between 15 to 25 wt % iota type carrageenan;
c) between 60 to 80 wt % lambda type carrageenan; and
wherein the carrageenan component comprises between 0.2 and 3 wt % of the topical formulation.

2. The topical formulation of claim 1 comprising:
between 8 to 12 wt % kappa type carrageenan;
b) between 17 to 23 wt % iota type carrageenan; and
c) between 65 to 75 wt % lambda type carrageenan.

3. The topical formulation of claim 1 wherein the kappa type carrageenan comprises greater than 80% kappa monomeric units; the iota type carrageenan comprises greater than 80% iota monomeric units, and the lambda type carrageenan comprises greater than 20% lambda monomeric units.

4. The topical formulation of claim 3 wherein the kappa type carrageenan comprises between 80% and 95% kappa monomeric units; the iota type carrageenan comprises between 80% and 95% iota monomeric units, and the lambda type carrageenan comprises between 15% and 30% lambda monomeric units.

5. The topical formulation of claim 1 that is a lotion, serum, or cream.

6. (canceled)

7. A method of tightening or smoothing the skin of a human comprising applying to the skin a topical formulation comprising a carrageenan component wherein the carrageenan component comprises:
a) between 7 to 12 wt % kappa type carrageenan;
b) between 15 to 25 wt % iota type carrageenan;
c) between 60 to 80 wt % lambda type carrageenan; and
wherein the carrageenan component comprises between 0.2 and 3 wt % of the topical formulation.

8. The topical formulation of claim 7 comprising:
a) between 8 to 12 wt % kappa type carrageenan;
b) between 17 to 23 wt % iota type carrageenan; and
c) between 65 to 75 wt % lambda type carrageenan.

9. The topical formulation of claim 7 wherein the kappa type carrageenan comprises greater than 80% kappa monomeric units; the iota type carrageenan comprises greater than 80% iota monomeric units, and the lambda type carrageenan comprises greater than 20% lambda monomeric units.

10. The topical formulation of claim 9 wherein the kappa type carrageenan comprises between 80% and 95% kappa monomeric units; the iota type carrageenan comprises between 80% and 95% iota monomeric units, and the lambda type carrageenan comprises between 15% and 30% lambda monomeric units.

11. The topical formulation of claim 7 that is a lotion, serum, or cream.

12. (canceled)

13. A method of cooling the skin of a human comprising applying to the skin a topical formulation comprising a carrageenan component wherein the carrageenan component comprises:
a) between 7 to 12 wt % kappa type carrageenan;
b) between 15 to 25 wt % iota type carrageenan;
c) between 60 to 80 wt % lambda type carrageenan; and
wherein the carrageenan component comprises between 0.2 and 3 wt % of the topical formulation.

14. The topical formulation of claim 13 comprising:
a) between 8 to 12 wt % kappa type carrageenan;
b) between 17 to 23 wt % iota type carrageenan; and
c) between 65 to 75 wt % lambda type carrageenan.

15. The topical formulation of claim 13 wherein the kappa type carrageenan comprises greater than 80% kappa monomeric units; the iota type carrageenan comprises greater than 80% iota monomeric units, and the lambda type carrageenan comprises greater than 20% lambda monomeric units.

16. The topical formulation of claim 15 wherein the kappa type carrageenan comprises between 80% and 95% kappa monomeric units; the iota type carrageenan comprises between 80% and 95% iota monomeric units, and the lambda type carrageenan comprises between 15% and 30% lambda monomeric units.

17. The topical formulation of claim 13 that is a lotion, serum, or cream.

18. (canceled)

19. A blend of carrageenans comprising:
a) between 7 to 12 wt % kappa type carrageenan;
b) between 15 to 25 wt % iota type carrageenan; and
c) between 60 to 80 wt % lambda type carrageenan.

20. The blend of claim 19 comprising:
a) between 8 to 12 wt % kappa type carrageenan;
b) between 17 to 23 wt % iota type carrageenan; and
c) between 65 to 75 wt % lambda type carrageenan.

21. The blend of claim 19 wherein the kappa type carrageenan comprises greater than 80% kappa monomeric units; the iota type carrageenan comprises greater than 80% iota monomeric units, and the lambda type carrageenan comprises greater than 20% lambda monomeric units.

22. The blend of claim 19 wherein the kappa type carrageenan comprises between 80% and 95% kappa monomeric units; the iota type carrageenan comprises between 80% and 95% iota monomeric units, and the lambda type carrageenan comprises between 15% and 30% lambda monomeric units.
